(19) United States
(12) Patent Application Publication
(10) Pub. No.: US 2007/0111976 A1
(43) Pub. Date: May 17, 2007

(54) Method of preventive on-demand hormonal contraception

(76) Inventors: Karin Schmidt-Gollwitzer, Berlin (DE); Gunter Stock, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/583,143

(22) Filed: Oct. 19, 2006

(30) Foreign Application Priority Data
Oct. 19, 2005 (DE) 102005050729.8

(51) Int. Cl. A61K 31/57; A61K 31/56

(52) U.S. Cl. 514/170

(57) ABSTRACT

The invention relates to a method of hormonal female controlled on-demand contraception in which a pharmaceutical preparation comprising at least one progestogen is administered transdermally on demand and on a single occasion prior to anticipated sexual intercourse.

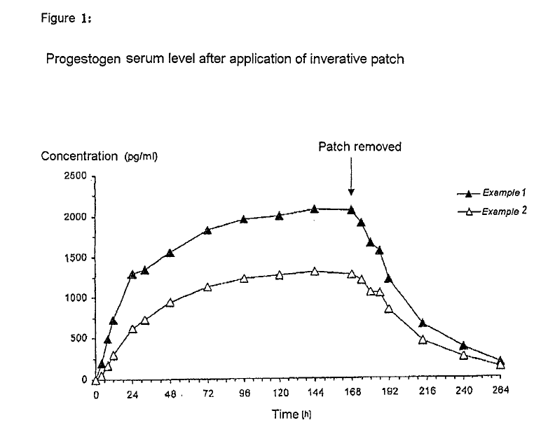

The present invention relates to a method of hormonal contraception, in which a pharmaceutical preparation comprising at least one progestogen is administered transdermally on demand and on a single occasion prior to anticipated sexual intercourse.

Hormonal contraception using low dosages of synthetic estrogens and progestogens administered orally each day is currently the most effective method of contraception.

Besides so-called combination products which comprise estrogen and progestogen, products comprising progestogens only are also available.

Administration of hormonal contraceptives is usually via the oral route. However, administration of progestogens as a depot preparation is additionally also possible. This includes injectable preparations, intrauterine pessaries and implants. Transdermal administration of an estrogen/progestogen combination released from a patch is also available on the market.

In European countries, the postcoital pill has been available since the 70s, likewise for the prevention of an unwanted pregnancy. It contains a high-dose combination of ethinylestradiol and progestogens.

For a few years, a high-dose progestogen product has been available which is administered orally within 72 hours in a single dose—or in divided doses with a second administration taking place within 16 hours after the first administration—after another contraceptive method has failed or after unprotected sexual intercourse.

The present invention is based on the object of providing a method of hormonal contraception which ensures higher contraceptive reliability in comparison with hormonal postcoital methods, is controlled by the woman and does not induce an abortion. Furthermore, it is intended to achieve higher tolerability than with other contraceptive agents.

The object is achieved according to the invention by a method of hormonal contraception in which a pharmaceutical preparation comprising at least one progestogen is administered transdermally on demand and on a single occasion prior to anticipated sexual intercourse. Higher tolerability is achieved by the lower dosage of the transdermally administered progestogen as compared with oral use.

Here, progestogens denote both the progesterone which is formed by the ovary in the second half of the cycle, and the synthetic derivatives having corresponding biological function with regard to inhibition of ovulation and maintenance of pregnancy.

The invention is based on the surprising realization that high contraceptive reliability can be achieved by precoital administration of the pharmaceutical preparation. If sexual intercourse does not occur, the patch can be easily removed again. In the event of sexual intercourse, administration of the progestogen over two or three days in a very low dosage has the following advantages:
High contraceptive reliability owing to the prolonged and more consistent duration of action as compared with oral administration.
Fewer side effects owing to the fact that the dose of progestogen or of estrogen/progestogen combinations is lower in comparison with oral administration.
One application of the patch will achieve the required active levels over a period of at least three days, that is protective contraception for at least three days.

The inventive method of hormonal contraception is of great significance for women who have intercourse only irregularly and/or rarely, two to three times a month, for example. On the one hand, it allows the woman or the pair to actively decide on contraception. On the other hand, ovulation is reliably inhibited with—in comparison to emergency contraception—a markedly lower dose, prolonged use and more consistent active levels. This treatment diminishes endometrial receptivity. This is a consequence of the precoital method being used, and therefore developing its effect, about 48 hours earlier than the postcoital method.

Frequency of use should where possible be restricted to two to three times per cycle, as more frequent use may induce cycle irregularities.

On-demand does not denote optional in this method of the invention. Rather, the woman must, in order not to become pregnant, use the inventive method in each case in which she can expect unwanted conception.

In the case of transdermal use, a single administration denotes attachment of an appropriately sized patch with defined release of progestogen over two or three days on a single occasion. If sexual intercourse does not take place, the patch is removed and the progestogen is substantially eliminated from the body within a short period of time. An adequate active progestogen level is reached after about 8 hours and stays at the same level for at least 48 hours with the patch remaining attached. This affords high reliability of the contraceptive method.

The progestogens most suitable for carrying out the invention are desogestrel, etonorgestrel, gestodene, levonorgestrel or trimegestone.

The amount of progestogen to be administered on a single occasion in the case of the precoital patch is for example about 50-100 μg of gestodene released within 24 hours from a patch 10 to 30 cm² in size, or an amount equivalent in effect of another progestogen.

The amounts of another progestogen equivalent in effect to the stated transdermal gestodene dose can be calculated on the basis of the amounts of oral prbgestogen required for inhibition of ovulation, taking into account the oral versus transdermal bioavailability of progestogens.

In the case of transdermal administration, the progestogen can be incorporated into a patch for example and thus be directly supplied to the blood circulation.

The patch must be of an appropriate size in order to ensure an adequate active level after use of the precoital patch. For gestodene, commensurate with a daily release of 50-100 μg, the patch has a size of about 10 to 30 cm², preferably of 10 to 20 cm².

According to the invention, additional inclusion of an estrogen component into the precoital patch is possible. Where an estrogen is additionally used in the patch, it is preferred to use ethinylestradiol in a dose which is sufficient for a daily release of 10 to 30 μg of ethinylestradiol.

The invention relates to the precoital patch per se for carrying out the method of the invention. The Claimed precoital patch here comprises exclusively one, or more progestogens as active substance, preferably gestodene. The daily release from this patch 10 to 30 cm², preferably 10 to 20 cm², in size is 50 to 100 μg of gestodene or an amount of another progestogen equivalent in effect to this amount of gestodene.

A pharmaceutical kit which comprises at least one transdermal patch comprising as active substance either only one progestogen or an active substance combination of progestogen and estrogen, preferably gestodene and ethinylestradiol, and a product information leaflet or instructions for use according to the inventive method, is also in accordance with the invention.

The invention is further explained below by means of exemplary embodiments from which further features, advantages and embodiments of the present invention become evident. These exemplary embodiments only serve as explanation. They therefore are in no way limiting to the protected subject matter of the present application.

### EXAMPLE 1

Production of a Patch to be Used in Accordance with the Invention

The precoital patch is produced as follows:

380 g of gestodene are stirred together with dioxane in a vessel and mixed until the substance has dissolved. This solution is transferred to a second vessel previously charged with 57.2 kg of the adhesive Arcare MA24A—composed of 68% heptane and 31% of the adhesive (1 to 25% of rosin ester and 75 to 99% of polyisobutylene)—and about 1% Irganox. The mixture is stirred and then applied to a release liner (polyester film 1876−75 μm, available from 4P, Forchheim, Germany) in such a way as to achieve a basis weight of 100 g/m². This layer is dried and then laminated with the backing layer (Cotran 9720, available from 3M, St. Paul, USA) . The layer thickness of the final product (laminate) is 100 μm. The patches having an area of 10 cm² are punched out from the laminate.

The resultant patch (10 cm²) has the following composition:

| | | |
|---|---|---|
| gestodene | 0.9 | mg |
| adhesive | 1.9 | mg |
| release liner | >10 | cm² |
| backing layer | 10 | cm² |

Using Durotak® as adhesive, for example, it is possible to produce further inventive patches in an analogous manner.

### EXAMPLE 2

When using 253 g instead of 380 g of gestodene (example 1), the resultant patch has the following composition:

| | | |
|---|---|---|
| gestodene | 0.6 | mg |
| adhesive | 1.9 | mg |
| release liner | >10 | cm² |
| backing layer | 10 | cm² |

### EXAMPLE 3

Determination of the Daily Release Rate

The patch is produced as described in example 1 or 2. The formulation is tested in the in-vitro mouse skin permeation test. The test is carried out using hairless mouse skin preparations (HsdCpb: NMRI-nu) from Harlan Bioservice for Science GmbH, Walsrode, Germany. The formulation is applied to the outside of the skin sample. The two are placed in a permeation measuring cell such that the inside of the skin contacts the receptor medium. HEPES buffer is used as receptor medium. Sodium azide is added to prevent germ growth and the receptor solution is heated to 32° C. Samples of the receptor solution are taken within defined time intervals and the gestodene concentration is determined by HPLC. The release rate is then determined as amount of active substance released per unit area and time [μg/cm²·24 h] using the calculated amounts of active substance.

The release rate measured in the in-vitro test is thus 30.9 μg/cm²24 h.

### EXAMPLE 4

Determination of the Resultant Hormone Level

One plaster each of example 1 or 2 was applied to the upper arm of healthy female volunteers, and the gestodene concentration in the serum was followed up on.

For this purpose, one aliquot of serum samples was extracted with ether, the ether layer was separated off and evaporated under a nitrogen atmosphere. The dissolved residue was incubated with rabbit anti-serum and 3H-labelled gestodene. Activated carbon was used to separate antibody-bound from unbound gestodene. The gestodene concentration was then determined radioimmunologically.

Depending on the concentration of gestodene in the patch, serum levels between 1500 and 2200 pg/ml (example 1) and 900 and 1300 pg/ml (example 2) were reached.

Reliably efficacious levels (>500 pg/ml) to ensure a contraceptive effect were achieved over 7 days. About 100 hours after removal of the patch, the serum gestodene level had fallen to the baseline value.

The features of the invention disclosed in the foregoing description and in the Claims can be essential, both individually and in any combination, for the realization of the invention in its various embodiments.

1. Method of hormonal female controlled on-demand contraception, characterized in that a pharmaceutical preparation comprising at least one progestogen is administered transdermally on demand and on a single occasion prior to anticipated sexual intercourse.

2. Method according to claim 1, characterized in that the pharmaceutical preparation is administered at the latest just prior to the first anticipated intercourse.

3. Method according to claim 2, characterized in that the pharmaceutical preparation is administered no earlier than 12 hours prior to intercourse.

4. Method according to claim 1, characterized in that the progestogen administered is selected from the group of compounds:
desogestrel,
etonorgestrel,
gestodene,
levonorgestrel or
trimegestone.

5. Method according to claim 4, characterized in that the progestogen is released daily in an amount which results in endogenous progestogen levels which are equivalent in effect to endogenous gestodene levels developing after daily transdermal administration of 50 to 100 μg of gestodene.

6. Method according to claim 5, characterized in that 50 to 100 μg of gestodene are administered daily.

7. Method according to claim 1, characterized in that an estrogen is administered in addition.

8. Method according to claim 7, characterized in that the estrogen administered is ethinylestradiol.

9. Method according to claim 8, characterized in that the ethinylestradiol is administered in an amount which brings about a daily release of 10 to 30 μg of ethinylestradiol.

10. Pharmaceutical preparation for use in any of the aforementioned methods according to claim 1.

11. Transdermal patch comprising exclusively one, or more progestogens as active substance.

12. Transdermal patch according to claim 11 comprising gestodene as progestogen.

13. Transdermal patch according to claim 11, from which an amount of progestogen equivalent in effect to 50 to 100 μg of gestodene is released.

14. Transdermal patch according to claim 11 having a size of 10 to 30 cm².

15. Transdermal patch according to claim 11, characterized in that the patch has a size of 10 to 20 cm² and that 50 to 100 μg of gestodene are released daily.

16. (canceled)

17. Pharmaceutical kit comprising at least one transdermal patch comprising one or more progestogens as active substance and optionally one or more estrogens, for carrying out a method according to claim 1, together with a product information leaflet describing the use of the patch in a method.
